(19) United States
(12) Patent Application Publication
(10) Pub. No.: US 2023/0372424 A1
(43) Pub. Date: Nov. 23, 2023

(54) ANTIVIRAL ACTIVE CINNAMON EXTRACT AND PROCESS

(71) Applicant: Novid 20 Ltd, Ma'ale Adumim (IL)

(72) Inventors: Eliezer J PINTHUS, Ma'ale Adumim (IL); Gilad AGMOM, Ramat Hasharon (IL); Abraham Jacob DOMB, Jerusalem (IL)

(21) Appl. No.: 18/246,911

(22) PCT Filed: Oct. 1, 2021
(86) PCT No.: PCT/IL2021/051186

§ 371 Date: Mar. 28, 2023

(30) Foreign Application Priority Data
Oct. 1, 2020 (IL) 277753
May 19, 2021 (IL) 283292

(51) Int. Cl. A61K 36/54; A61K 9/14; A61P 31/16

(52) U.S. Cl. CPC A61K 36/54; A61K 9/14; A61P 31/16

(57) ABSTRACT

The present invention concerns a polyphenol extract from cinnamon bark in the form of nanoparticles and its use as an antiviral agent.

### TECHNOLOGICAL FIELD

This present invention relates to a cinnamon extract and a process for obtaining the extract by natural water extract of Cinnamon Cassia bark in large scale.

### BACKGROUND ART

References considered to be relevant as background to the presently disclosed subject matter are listed below:
WHO Influenza (seasonal) factsheets 2018, available at: https://www.who.int/news-room/fact-sheets/detail/influenza-(seasonal)
Killbourne, E. D., Zhang, Yan, B., Lin, Mei-Chen 2006 “Influenza pandemic of the 20^{th} century” Emerging Infections Diseases (Centers for Disease Control and Prevention) 12 (1), 299
Premanathan et al, Indian J Med Res . 2000 September; 112:73-7.
Fink, R. C., Roschek, B., Alberte, R. S., 2009 International Medical Press 2040-2066
Barak, I., Ovadia, M. Natural inhibitor of influenzaA-PR8 extracted from cinnamon. 18^{th} ICAR meeting, Apr. 11-14, 2005, Barcelona, Spain. Antiviral Research Vol 65, A65
Ovadia, M., Kallily, Y., Bernstein, E. 2009. Cinnamon fraction neutralizes avian influenza H5N1 both in-vitro and in-vivo. 22^{nd} ICAR meeting, 3-7 May 2009, Miami Beach, Florida. Antiviral Research Vol. 82(2), A35
Sevillia, G. Kamensky, M., Finger, A., Ovadia, M. 2007. Cinnamon Extract Inhibits Avian Influenza H9N2 Both in-vitro and in-vivo. Options for the control of Influenza VI., p. 467-469
Munazza, F., Zaidi, N. S., Amraiz, D., Afzal F. 2016 In Vitro Antiviral Activity of Cinnamomum cassia and its Nanoparticles Against H7N3 Influenza A Virus. J. Microbiol. Biotechnol. (2016), 26(1), 151-159
Kallily, I. Kamensky, M. Finger, A. Ovadia, M. 2010. Immunization against Newcastle disease virus using cinnamon fraction. Modern Veterinary Vaccines and Adjuvants (MVVA) conference, November 17-19, Budapest Hungary.
Gueta, K., Kamensky, M. Finger, A., Ovadia, M. 2008. Immunization against Newcastle Disease Virus Using Cinnamon Fraction. Annual meeting of Veterinary Institute Bet-Dagan. Israel Journal of Veterinary Medicine Vol. 63(2)
Klaus A ., Wöhrlin F., Lindtner O., Heinemeyer G., Lampen A. 2010
Toxicology and risk assessment of coumarin: focus on human data. 2010
Mol. Nutra Food Res.2010 February; 54(2):228-39.

Acknowledgement of the above references herein is not to be inferred as meaning that these are in any way relevant to the patentability of the presently disclosed subject matter.

### BACKGROUND

Various Viral infections have major impact on global health. Despite the availability of vaccines, Influenza is still associated with significant morbidity and mortality. The World Health Organization estimates that worldwide, annual influenza epidemics result in about 3-5 million cases of severe illness and about 290,000 to 690,000 deaths (Influenza (Seasonal) World Health Organization 2018).

Few viral pandemics outbreaks occurred during the last century at about 10 years intervals (Killbourne et al. 2006), requiring development of new vaccines, or modification of existing vaccines to fit new strains. Currently the COVID 19 outburst significantly affects the everyday life of every human being! The novel coronavirus (COVID-19) pandemic is a major medical challenge with high morbidity and mortality. The high rate of infection, together with significant progression rates to acute respiratory distress syndrome (ARDS) and pneumonitis, places high stress on the global medical and economic communities. Due to the anticipated long development time, vaccines, which are major weapon in disease prophylaxis, are not yet available. Moreover, vaccines, even when available, are usually much less efficacious for older population and the immune-compromised patients. They are relatively expensive, require refrigeration in storage and transportation, and are therefore hardly applicable in developing countries and isolated territories.

Alternative approach to the development of a new vaccine, is separation and isolation of compounds which are already available in nature. More specifically—Plant sources with proven and active anti-viral and anti-inflammatory properties.

Cinnamon has been valued for its medicinal properties for thousands of years, and is classified as a medicinal plant in many countries around the world. The ground bark powder and its extracts are consumed broadly both as spice and as food supplement. Modern science supports the following Cinnamon properties: (a) loaded with powerful antioxidants such as polyphenols; (b) the antioxidants in cinnamon have anti-inflammatory effect; (c) cinnamon can dramatically reduce insulin resistance; (d) cinnamon reduce blood sugar levels, having a potent anti-diabetic effect. Further, cinnamon exhibit significant antiviral properties: (1) a laboratory study looking at HIV-infected cells found that cinnamon was the most effective treatment of all 69 medicinal plants studied, (Premanathan et al,. 2000); (2) test-tube studies have shown that cinnamon can help fight HIV-1, the main type of HIV virus in humans, (Fink et al, 2009); (3) cinnamon aqueous extract showed ability to inhibit various strains of Influenza A including H1N1 (Barak & Ovadia, 2005), Avian influenza H5N1 (Ovadia et al 2009), H9N2 (Sevillia et al, 2007), H7N3 (Munazza et al, 2016); (4) cinnamon aqueous extract exhibited antiviral activity on Newcastle virus (Kallily et al, 2010) and Sendai virus (Gueta & Ovadia, 2005); (5) high molecular weight Cinnamon extract performed in large variety of viral applications as demonstrated by M. Ovadia in U.S. Pat. No. 9,364,511 (2006). Cinnamon High Molecular Weight Fraction enables a unique inactivation of enveloped viruses and can be applied in the prevention/treatment of specific viral infections. The pre-clinical work suggest that this Cinnamon extract immediately inhibits viral activity by adhering to the viral envelope. This process is not membrane destructing, but rather the virus remains intact, enabling the immune system to recognize it and produce protective antibodies.

### GENERAL DESCRIPTION

The preset invention is directed to a cinnamon extract, in particular, a polyphenol extract from cinnamon bark in the form of nanoparticles and to an improved large-scale process for obtaining the extract that was shown to exhibit broad anti-viral properties. The polyphenol extract is characterized by its molecular weight, water solution, viscosity and its particle size when measured by Dynamic Light Scattering (DLS) and/or Transmission electron microscopy (TEM). The polyphenol extract is obtained solely by physical separation processes with no chemical reactions or chemical additives. Thus, the present invention is directed to a polyphenol extract from cinnamon bark that is slightly water soluble where the water solution indicates a nano-dispersion characterized by;
having a molecular weight of less than 10 KD when determined by aqueous Gel Permeation Chromatography (GPC);
intrinsic viscosity in water of 0.1-1.0 using Ubbelohde viscometer.

The polyphenol extract according to the present invention is in the form of nanoparticles in the size of 100-500 nanometer when determined by Dynamic Light Scattering (DLS) and/or Transmission electron microscopy (TEM). Preferably, the particle size may be in the range of 100-170 nanometer.

The polyphenol extract preferably has a molecular weight in the range of 3-6 KD, most preferably about 2-4 KD when determined by gel permeation chromatography GPC. The intrinsic viscosity is 0.25 or less.

The present invention is further directed to a cinnamon extract obtained by a process comprising:
mixing cinnamon powder, pulp or flakes with an aqueous phosphate buffer solution to obtain an aqueous solution that is agitated;
centrifuging the aqueous solution to obtain a supernatant;
heating the supernatant followed by cooling;
passing the supernatant through a GAP filter to obtain a crude extract;
exposing the crude extract to ultrafiltration or nanofiltration having a cutoff membrane of 1 KD to 10 KD and collection the retentate having a volume of about 10% of the crude extract;
optionally re-exposing the retentate of the ultrafiltration or nanofiltration process to a second round;
concentrating the obtained retentate or combined retentate to obtain a liquid or powder product.

The present invention is yet further directed to a polyphenol extract or a cinnamon extract and its use as a broad antiviral agent. The antiviral agent in accordance with the invention is used for treating in particular infections caused by an enveloped virus by inhibiting viral replication. The polyphenol extract or cinnamon extract may be in the form of an aqueous solution that may comprise a buffer, preferably a phosphate buffer. Alternatively, the polyphenol extract or a cinnamon extract may be formulated into lozenges, candy, foods such as ice cream and beverages, may be incorporated into compressed tablets or loaded in hard shell capsules for oral intake, or may be dispersed in water for nasal spray or ocular delivery to treat viral infections.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A: provides pictorial demonstrations of the cinnamon extract (a)—powder; (b)—supernatant; (c)—diluted supernatant; (d)—lyophilized particulate of the extract.

FIG. 1B: demonstrates the interaction of the cinnamon extract with FeCl₃ demonstrating the presence of hydroxyls.

FIG. 2 provides a FT-IR analysis of the cinnamon extract.

FIG. 3 provides Gel Permeation Chromatography (GPC) of the cinnamon extract.

FIGS. 4A and 4B provide Transmission Electron Microscopy (TEM) images of the cinnamon extract.

FIG. 5 demonstrates Hemagglutination inhibition by Test Item P8150 at several concentrations of P8150 with H1N1 influenza virus.

FIG. 6 demonstrates Hemagglutination inhibition by Test Item L2703 at several concentrations of L2703 with H1N1 influenza virus.

FIG. 7 demonstrates Hemagglutination inhibition calculation.

### DETAILED DESCRIPTION OF EMBODIMENTS

The present invention makes use of cinnamon, preferably cinnamon cassia for extracting at least one fraction of the cinnamon previously found to have antiviral activity by a process that employs membrane technology. The resultant extract product is polyphenol nanoparticles characterized by shape, size, molecular weight, viscosity. The dry weight basis (DWB) of the polyphenol in the extract is between 50 and 95, in particular 70-95, most preferably 80-93. The membrane technology according to the present invention includes filtration with organic or mineral membranes, membranes with a variety of cutoff sizes to be used at different stages of the process. In particular, the present invention makes use of ultrafiltration or nanofiltration of an aqueous or buffer extraction of crude cinnamon or an ultrafiltration of an aqueous or buffer extraction of a previously CO₂ supercritical or non-polar organic solvent extraction of cinnamon, to obtain the polyphenol nanoparticles. Non-limiting examples on non-polar organic solvent are CH₂Cl₂, hexane, acetone, ethyl acetate.

The process of extraction according to the present invention is conducted without the involvement of any chemical reagents other than the aqueous solution or the aqueous buffer. The cinnamon source may be natural cinnamon bark, waste or residue of the flavor industry, that is either de-oiled and/or de-fatted cinnamon pulp, flake, or powder or did not undergo a process of de-oiling or de-fatting. The improved process of cinnamon extraction that can use a source of cinnamon that avoids or reduces significantly undesired compounds, more specifically coumarins. The process comprises:
mixing cinnamon powder, pulp or flakes with an aqueous phosphate buffer solution to obtain an aqueous solution that is agitated;
centrifuging the aqueous solution to obtain a supernatant;
heating the supernatant followed by cooling;
passing the supernatant through a GAP filter to obtain a crude extract;
exposing the crude extract to ultrafiltration or nanofiltration having a cutoff membrane of 1 KD to 10 KD and collection the retentate having a volume of about 10% of the crude extract;
optionally re-exposing the retentate of the ultrafiltration or nanofiltration process to a second round;
concentrating the obtained retentate or combined retentate to obtain a liquid or powder product.

According to the present invention cinnamon bark whether crude cinnamon or waste from the flavor industry, is either grounded or subject to CO₂ supercritical or non-polar organic solvent extraction of dried bark cinnamon cassia to obtain pulp, flakes, and the pulp or flakes are mixed with an aqueous solution that may be an aqueous solution comprising a buffer, preferably phosphate buffer. The cinnamon can be mixed with a cold or hot aqueous solution or under ambient conditions. The phosphate buffer when used is in a concentration of 0.01-0.05M, preferably, 0.02M, 0.03M or 0.04M. The ratio between the cinnamon source in any of its forms and the aqueous solution water comprising the buffer or not buffer being in the range of 1:3-50, preferably 1:5-1:30. The solution is mixed for a period of several hours. The mixture is stirred at ambient temperature, or under cold or hot conditions. The mixture is stirred for a period between 3 and 30 hours, preferably, 3, 4, 5, 6, 7, 8, 10 to 20, 21, 22, 23, 24 or 30 hours at ambient temperature. The mixing of the cinnamon material and the buffer may also be a two-step process, wherein initially the cinnamon source, is mixed with the buffer at a ratio of 1:4-1:7, preferably 1:5 in ball mill for a period 2-5, preferably 3 hours followed by further dilution to a ratio of 1:18-1:22, preferably 1:20 and agitated for additional 5-8, preferably 6 hours.

In order to obtain better extraction by means of higher yield, for controlling the viscosity or the sedimentation, the aqueous extraction is can also be conducted at hot or cold processing. It can also utilize enzymatic treatment, non-limiting enzymes that may be used are lipase, Cellulases, Hemi-cellulases. The use of enzymes according to the present invention can be done at several steps for either improving and obtaining a mor clear solution (removing cloudiness) or for modifying the brix index. For increasing the yield, the process according to the present invention can also make use and include one or more stages of counter-current multistage rinsing/washing.

Following the aqueous extraction, a cloudy solution is obtained including sediments. In order to lower the colloidal sedimentation, the resultant mixture of aqueous extract and solids/sediments is subjected to a process of separating the aqueous extract form the solids/sediments. Such separation may be done by either centrifugation or decantation or a process of de-pulping. It should be noted that after decantation, the resultant clearer aqueous extract may be subject to centrifugation. Such centrifugation of de-pulping can be conducted at a temperature range of 4° C. to just below boiling temperature, i.e. about 98° C. Following this process of sedimentation, the resultant more clear (semi-clear) supernatant solution is characterized by its cloudiness, i.e. its Brix index. Preferably, the Brix index is in the range of 1-6 variations arising from contradicting requirements of the final product. The concentration of the extracted active component and efficiency of the process can dictate varying indexes that affect the overall yield, duration and cost of the process.

The supernatant is pasteurized by heating to about 85° C. for a period of about 0.5-5 minutes. Following the pasteurization, the supernatant is cooled to any temperature in the range of 4° C.-60° C.

The resultant aqueous supernatant solution is filtered to remove more fine particles left after the above process for lowering the colloidal sedimentation. The aqueous supernatant solution is then optionally subjected to filtration using a 5-10 micron GAP filter at the above indicated temperature, i.e. 4° C.-60° C. The resultant filtered or non-filtered solution is subject to a process of ultrafiltration or nano membrane filtration. These two processes are conducted such as to increase the yield of desired fractions characterized by particles size, and/or weight that include the desired active component or components. Thus, in accordance with the present invention the membrane cutoff is pre-defined and set to let particles with 1.0 KD to 10 KD, preferably 3-6 KD, most preferably 5 KD. The discarded permeate enable withdrawal of the undesirable compounds, while in the retentate the concentration of the valuable separated molecules having the desired size and/or weight is being built up resulting in a concentrate having a considerable amount of desired active compounds. In accordance with the present invention and in order to further increase yield of the active fraction, the permeate can be subject to a nanofiltration the result of which is combined with the retentate. Non-limiting membranes are that may be used in the membrane separation technology are spiral membrane cartridges, tubular cartridges, hollow fiber cartridges, flat sheet plate and frame, all in the desired particle size range. The resulting (membrane) concentrated retentate has a Brix index in the range of about 3-20. One may repeat the ultrafiltration or nano membrane filtration so as to further concentrate the retentate. Alternatively, and in order to further increase yield, the retentate may be subject to defiltration, that is rinsing the retentate with water to remove small molecules. Alternatively, or additionally, the retentate may be subjected to a further concentration by thermal evaporation under ambient pressure or vacuum.

Products could be aseptically packaged for delivery in liquid form or in other secured packaging forms complying with storage temperature. The extract may be further dried to powder preferably by freeze drying.

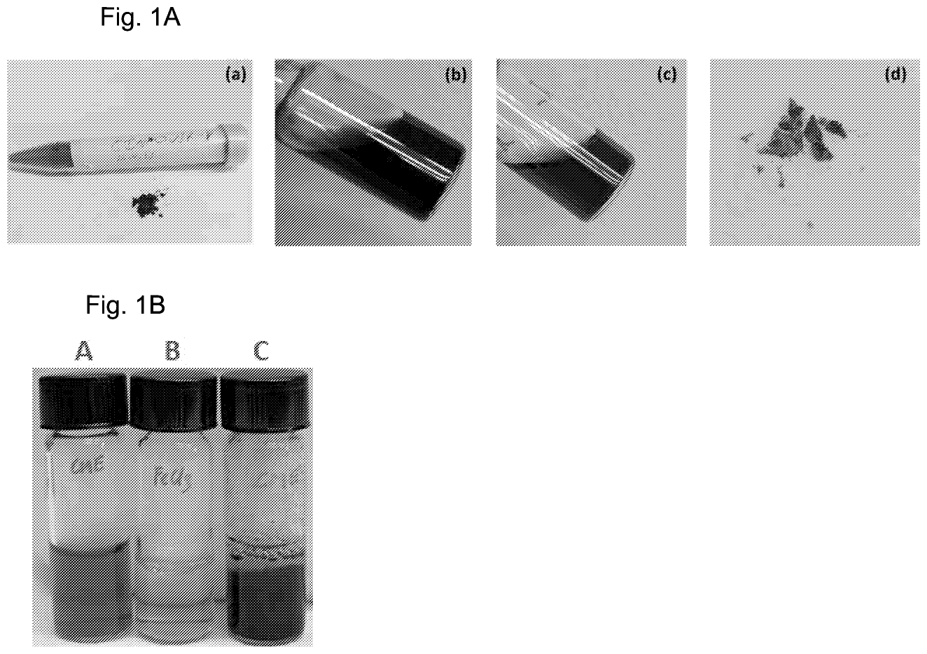

The polyphenol extract was obtained as dry dark brown fine powder having slightly solubility in water (FIG. 1A). Turning in particular to FIG. 1A, are given four appearances of the extract: (a) Cinnamon extract powder that was obtained by the process of the present application; (b) the supernatant concentrate; (c) diluted supernatant; (d) lyophilized water-soluble fraction of the cinnamon extract. The color and appearance of the supernatant and the lyophilized material that were obtained for both untreated (water, no pH adjusted) and treated (pH adjusted to 10 using NaOH) cinnamon extract remain similar.

The presence of phenolic group was assessed by the reaction of the dried extract with FeCl₃ and the color change from brown to purple associated with the interaction (FIG. 1B). Turning to in particular to FIG. 1B, are given (A) the cinnamon extract dissolved in water; (B) an aqueous FeCl₃ solution; (C) the result of the addition of the aqueous FeCl₃ to the cinnamon extract that gives the dark greenish violet color precipitate indicative of the presence of phenolic group.

Further, when the cinnamon extract heated to 300° C., some part was melted and the other part is not. This is due to the presence of mixture of organic and inorganic compounds. The presence of a rather high percentage of oxygen is also confirmed by the elemental analysis given below (Example 1).

The extract powder of this invention is farther formulated into lozenges, candy, and functional foods such as ice cream and beverages. The particles may be incorporated into compressed tablets or loaded in hard shell capsules for oral intake. The cinnamon extract powder may be dispersed in water for nasal spray or ocular delivery to treat viral infections.

The term “about” as used throughout the application is understood as the indicated value having a range of ±10%.

The following experiments were done for characterization of the polyphenol extract obtained by extraction from cinnamon and determining its properties. Generally, the extraction is done by the methods that are described in IL application No. 277753 incorporated herein by reference.

### EXAMPLES

### Extraction

### Example 1

10 kg of cinnamon cassia ground bark powder, were charged to an agitated vessel together with 200 kg of 0.02M phosphate buffer at pH 6.98, and mixed for 20 hours. The bulk extract was discharged through centrifuge and collected in another vessel. The extract was heated to 85° C. and kept for 5 minutes followed by cooling down to 30° C. The aqueous extract at 1.1 Bx was sent through GAP filter equipped with 10-micron filter bag, and collected in the UF feeding tank. The liquid extract (175 liter), was then fed to the hollow fiber 10 KD (M.W.) cut off UF membrane at 30 C. Processing mode was batch type recirculated process. During the UF process the retentate was concentrated by 10-fold down to 10% of the original stock (17 liter). 90% was discarded as permeate. Both the permeate and retentate Brix were measured and determined to be 0.9 and 4.0 respectively.

The retentate was passed again through the same UF membrane, but shortly afterwards rate slowed down and the UF process was stopped once permeate removal declined to insignificant dripping. Retantate collected (12 liter) was at 6.1 Bx. This retentate was concentrated by evaporation to 15 Bx and then discharged. Product was freeze dried and ground to powder. Sample P8150 (see anti-viral activity) was taken

### Example 2

30 kg of dry pulp flakes obtained from solvent extraction of cinnamon cassia bark, containing 25 mg/kg of Coumarin and less than 5 ppm Dichloromethane were charged to a Macintyre mill together with 150 kg of 0.02M phosphate buffer at pH 6.94. Milling was done at 20° C. for 3 hours. The slurry was discharged and added into agitated vessel containing 400 liters of phosphate buffer and mixed for 6 hours. The bulk extract was discharged through centrifuge and collected in another vessel. The extract was heated to 85° C. and kept for 5 minutes followed by cooling down to 30° C. The aqueous extract at 1.2 Bx was sent through GAP filter equipped with 10-micron filter bag and collected in the UF feeding tank. The liquid extract was then fed to a hollow fiber 10 KD (M.W.) cut off UF membrane at 50° C. Processing mode was batch type recirculated process. During the UF process the retentate was concentrated by 10-fold down to 10% of the original stock. 90% was discarded as permeate. Both the permeate and retentate Brix were measured and determined to be 0.8 and 4.2 respectively. Second UF run was much more efficient than in example 1 above, most probably due to the specific raw material lacking fatty substance which tend to coat the membrane. The final retentate amount was 7.6 liter at 11.3 Bx. Sample L2703 was taken from this fraction (see anti-viral activity). Part of the product was freeze dried and ground to obtain free flowing powder. Both liquid and powder parts were analyzed for total phenol content (TPC), frequently expressed as the content of the galic acid. The result was 90.4% dry weight basis (DWB) where the initial TPC was 26.2%—undoubtedly a significant increase of the TPC.

### Example 3

30 kg of dry pulp flakes obtained from CO₂ extraction of cinnamon cassia bark, containing 16 mg/kg of Coumarin, were charged to an agitated vessel together with 570 liters of 0.02M phosphate buffer at pH 6.89. The crude mixture was agitated for 24 hours. The bulk extract was discharged through centrifuge and collected in another vessel. The extract was heated to 85° C. and kept for 5 minutes followed by cooling down to 40° C. The extract was sent through GAP filter equipped with 10-micron filter bag. Liquids at 1.2 Bx fed to the hollow fiber 5KD (M.W.) cut off UF membrane. Processing mode was batch type recirculated processing. Concentrating the retentate up to 12 Bx. The product was further concentrated by heat exchanger. Once the retentate reached 20Bx it was discharged. The product was analyzed for polyphenol content and the result was 18.4% DWB.

### Example 4

In order to enable extraction of 100% natural product, the cinnamon cassia bark was initially subject to CO₂ flavor extraction and the residue was subjected to an aqueous extraction. Hence 1 kg of dry pulp flakes obtained from CO₂ extraction of cinnamon cassia bark, containing 16 mg/kg of Coumarin, were charged to an agitated vessel together with 25 liters of tap water. The crude mixture was agitated for 15 hours. pH was determined to be 6.2 and the bulk extract was discharged through centrifuge and collected in another vessel. The extract was heated to 85° C. and kept for 5 minutes followed by cooling down to 25° C. The extract was sent through GAP filter equipped with 10-micron filter bag. Liquids at 1.2 Bx fed to the hollow fiber 10 KD (M.W.) cut off UF membrane. Processing mode was batch type recirculated processing. Concentrating the retentate up to 13 Bx. At this stage product was discharged. The product was analyzed for polyphenol content and the result was 91.1% DWB.

### Example 5: Elemental Analysis of Cinnamon Extract

Elemental analysis of cinnamon extract shows the percentages of carbon, hydrogen and nitrogen are: 52.52%, 4.64% and 0.00% N, respectively. In addition, the results revealed that the absence of nitrogen atoms. These results clearly indicate the presence of a high percentage of oxygen atoms, which confirm the polyhydroxy compound.

### Example 6: Solubility Measurement

The solubility test for the cinnamon extract powder was performed in water (no pH adjustment) and at pH 10 (pH adjusted using NaOH). 100 mg of the material was dispersed in the 3 mL of the aqueous medium and kept on overnight mixing at high speed. Thereafter, the aqueous mixture was centrifuged at 10,000 rpm for 30 min at 4° C. Both the precipitate and the supernatant (soluble fraction) were collected in separate vials and lyophilized.

The water/solvent solubility of the cinnamon extract was determined by adding 100 mg of the extract powder in 1 ml of solvent or water at pH 7 or pH 10. After 24 hours of agitation, the mixture was centrifuged at 4000 rpm for 10 min. and the clear supernatant was isolated and lyophilized. The soluble part was 30 mg and the insoluble part was 70 mg (3% solubility). Among organic solvents, cinnamon extract had some solubility in DMSO (0.7%) and sparingly soluble in ethanol and chloroform. The solubility of cinnamon extract in various solvents is given in Table 2. The clear supernatant was in fact a dispersion of nanoparticles of 145-180 nm as determined by particle size analyzer, Dynamic Light Scattering (DLS).

TABLE 2
Solubility of cinnamon extract in various solvents

| S. | | |
|---|---|---|
| No. | Solvent | Solubility* |
| 1 | Water (DDW, | 3.0% |
| | pH7 or pH10) | |
| 2 | DMSO | 0.7% |
| 3 | Ethanol | Sparingly soluble (<0.2%) |
| 4 | Chloroform | Sparingly soluble (<0.2%) |

### Example 7: Dynamic Light Scattering (DLS) and Zetapotential Measurement

The diluted sample of soluble fraction of the cinnamon extract in water was tested for any presence of nanoparticulate using DLS. The results reveal that the soluble fraction of the cinnamon extract which was prepared without any pH treatment showed the presence of nanoparticulate with size of 148.6±23.5 nm. On the other hand, the soluble fraction of the cinnamon extract which was prepared with pH treatment showed the presence of nanoparticulate with size of 187.4±110.4 nm. The zeta potential data reveal a high negative charge on the surface of the nanoparticles (Table 3). If the nanoparticles in suspension have a large negative potential, then they will tend to repel each other and there will be no tendency for the particles to come together. Therefore, we did not observe any sedimentation in the samples when kept undisturbed over time at room temperature. The surface charge was simultaneously determined using the same diluted solution. DLS and zetapotential was performed using Zetasizer (Nano ZS, Malvern Instruments, UK) equipped with inbuilt software. Particle size analysis of the clear supernatant indicated that the extract is in the form of nanoparticles of 100-200, preferably at 120-150 nm. This indicate that the compound is not water soluble but insoluble nanoparticles of the compound. The results are given in Table 2 below.

TABLE 3
DLS and surface charge determination of the soluble
fraction of cinnamon powder in water.

| | Size | | Zeta |
|---|---|---|---|
| | (nm)/ | | potential |
| Sample | DLS | PDI | (mV) |
| Soluble fraction | 148.6 ± 23.5 | 0.403 ± 0.124 | −22.2 ± 5.90 |
| obtained without | | | |
| treatment | | | |
| Soluble fraction | 187.4 ± 110.4 | 0.473 ± 0.110 | −22.2 ± 7.41 |
| after NaOH treatment | | | |

### Example 8: Example FT-IR Analysis of Cinnamon Extract

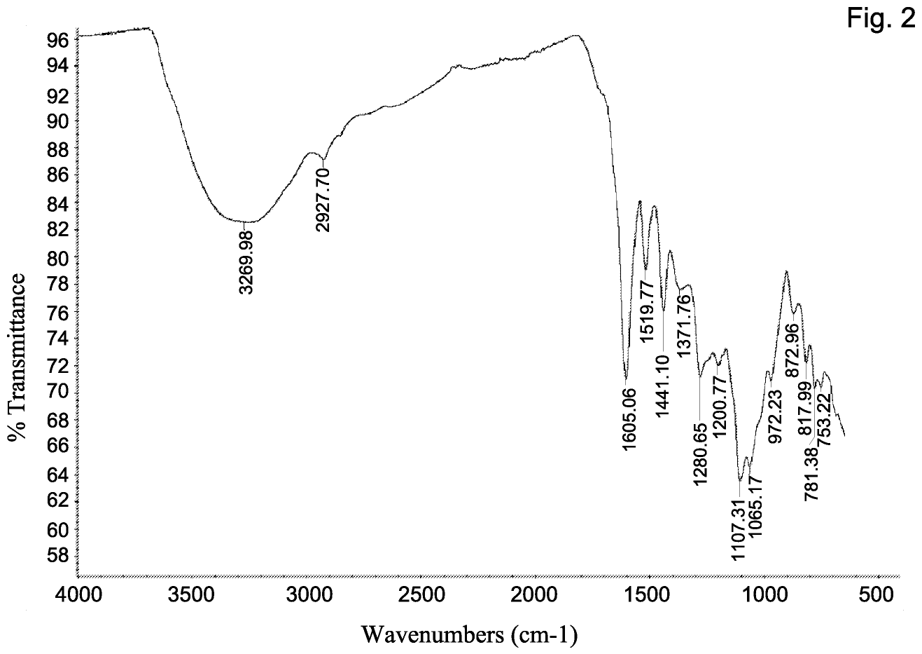

FT-IR spectrum was obtained in neat condition using a Smart iTR ATR sampling accessory for Nicolet iS10 spectrometer with a diamond crystal (Thermo Scientific, Massachusetts) and given in FIG. 2. The broad frequency observed at 3269 cm⁻¹ confirms the hydroxy groups present in the extract. The band detected at 2927 cm⁻¹ confirms the aliphatic C—H stretching frequency. The bands seen in the region of 1605 cm⁻¹ and 1107 cm⁻¹ confirm the C═C and C—O stretching frequencies respectively.

Further an NMR analysis of cinnamon extract was also done as follows further confirming the presence of both aromatic and hydroxyl groups. ¹H NMR spectra was obtained on a Varian 300 MHz spectrometer, in tubes with 5 mm outside diameters. D₂O or DMSO-d₆ served as a solvent and shift reference. The ¹H NMR spectra of cinnamon extract in DMSO-d₆ and D₂O was determined. The NMR spectra shows the presence of a broad peak in the range of 7.47-9.70 ppm confirming the presence of aromatic protons. The broad peaks in the range of 4.00-6.00 ppm exhibit the presence of hydroxyl groups. The peaks at 1.24 ppm and 3.75 ppm confirms the presence of aliphatic protons.

### Example 9: TEM Measurement

Transmission electron microscopy (TEM) was used to understand the exact shape of the nanoparticles. The TEM analysis was performed using JEOL JEM-1400Plus by applying ˜10 μL of samples resuspended in DDW to a 200- or 400-mesh copper grid covered by carbon-stabilized Formvar film (SPI, West Chester, PA). The samples were dried overnight before scans were performed. The nanoparticles were sequentially stained with negative stain NanoVan® (Methylamine Vanadate) and the images were analyzed using inbuilt software (SoftImaging System GmbH, Münstar, Germany).

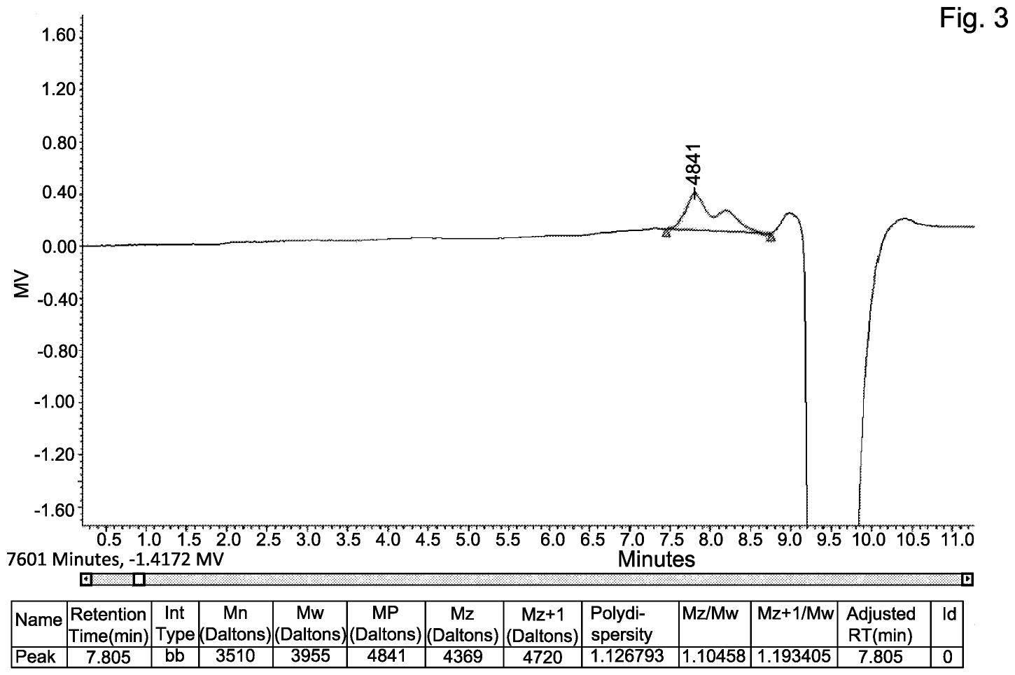

Turning to FIG. 3, TEM measurements indicate that the nanoparticles were nearly spherical. As shown in FIG. 2A, the nanoparticles of the fraction that was obtained without any pH treatment was more spherically shaped than compared to the soluble fraction that was obtained after the NaOH treatment FIG. 2B. Overall, the size obtained from the DLS data matches with the TEM images for the nanoparticles.

### Example 10: Viscosity of the Cinnamon Extract

The water solubility of the cinnamon extract was determined at room temperature by adding 100 mg of the extract powder in 1 ml of water or pH 10 solution. After 24 hours the mixture was centrifuged and the clear supernatant was isolated that contain 30 mg/ml. Viscosity measurement of the supernatant indicted low viscosity of 0.23 (for 1% in water 25° C. using Ubbelohde viscometer).

### Example 11: GPC Analysis of Cinnamon Extract

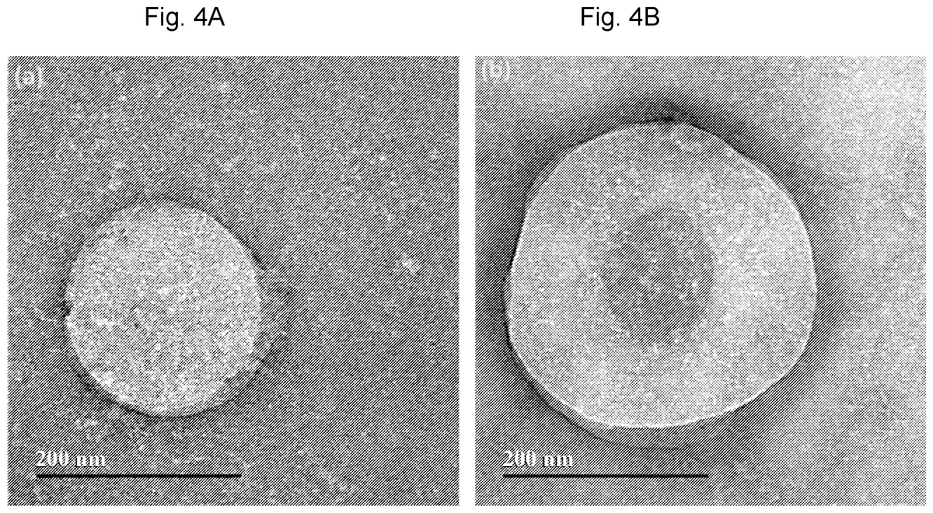

The molecular weight of cinnamon extract was estimated by gel permeation chromatography (GPC) system and eluted with water. Molecular weight was determined relative to pullulan standards with a molecular weight range of 500-100,000 Da. The molecular weight of the cinnamon extract is ˜4,000 Da (FIG. 4).

### Anti-Viral Activity

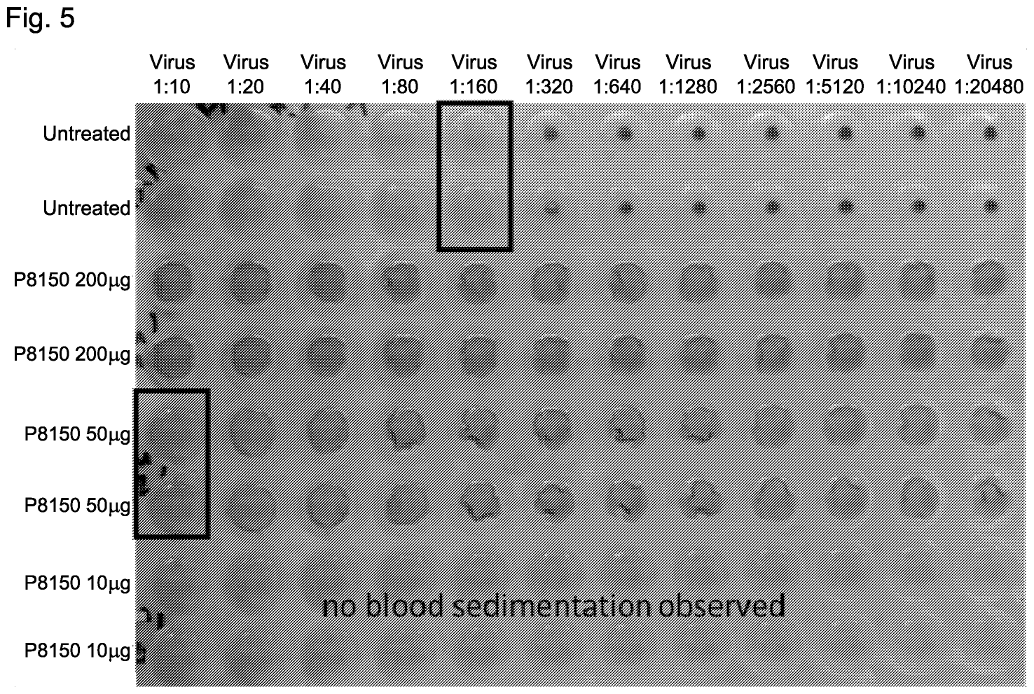

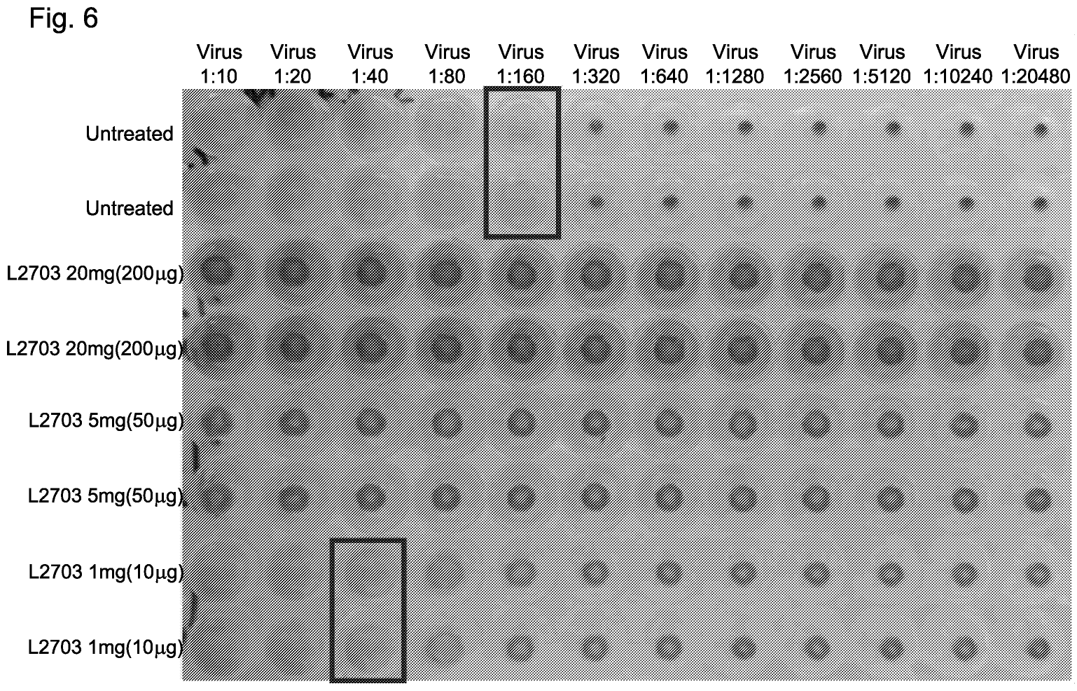

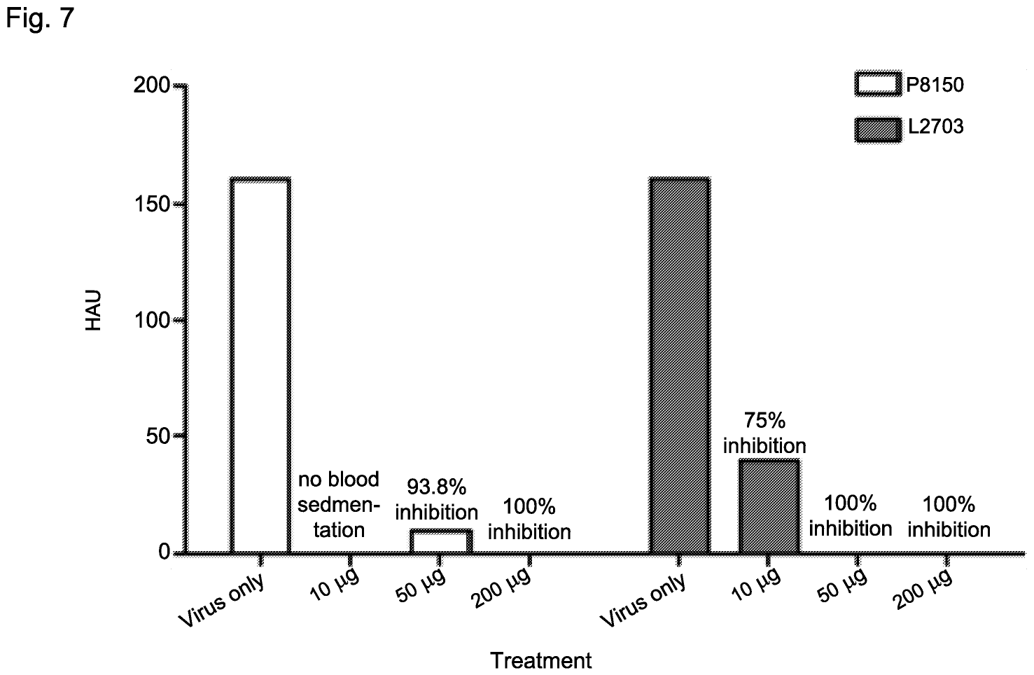

The anti-viral activity of compositions comprising the products obtained by the extraction processes according to the present invention was tested. FIG. 5 describes the inhibition of Hemagglutination inhibition by Test Item P8150 obtained by the process of the present invention. H1N1 influenza virus was pre-incubated with P1850 at elevating concentrations before chicken RBCs were added. The hemagglutinating unit (HAU) was determined as the smallest amount of virus that still prevented precipitation of the RBCs from forming a ring at the bottom of the test tube. Red squares indicate 1 HAU. FIG. 6 describes Hemagglutination inhibition by Test Item L2703 obtained by the process of the present invention. H1N1 influenza virus was pre-incubated with the L2703 at elevating concentrations before chicken RBCs were added. The hemagglutinating unit (HAU) was determined as the smallest amount of virus that still prevented precipitation of the RBCs from forming a ring at the bottom of the test tube. Red squares indicate 1 HAU. FIG. 7 provides calculations of the hemagglutination inhibition. H1N1 influenza virus was pre-incubated with the TIs at elevating concentrations before chicken RBCs were added. The hemagglutinating unit (HAU) was determined as the smallest amount of virus that still prevented precipitation of the RBCs from forming a ring at the bottom of the test tube.

### Example 12

Two Test Items (TIs), P8150 (Example 1) and L2703 (example 2) were tested for their ability to inhibit H1N1 influenza virus hemagglutination. Both TIs were pre-incubated with the virus prior to the addition of chicken red blood cells (RBCs).

FIGS. 5 and 7 display results evidencing that P8150 completely inhibited H1N1 mediated hemagglutination at 200 μg (100% inhibition), and almost completely inhibited H1N1 mediated hemagglutination at 50 μg (93.8% inhibition or HA inhibition by 16). At 10 μg no blood sedimentation occurred and hemagglutination inhibition could not be calculated. It is possible that at 10 μg the TI P8150 either affects hemagglutination by itself (or somehow prevents blood sedimentation) or improves H1N1 virus mediated hemagglutination.

FIGS. 6 and 7 display results evidencing that L2703 completely inhibited H1N1 mediated hemagglutination at 20 mg (200 μg) (100% inhibition) and at 50 mg (50 μg). At 1 mg (10 μg) L2703 inhibited H1N1 mediated hemagglutination by 75% or HA inhibition by 4.

As the TIs were incubated directly with a viral stock solution (and not with the virus producing cells) it is likely that the TIs directly prevented the virus from attaching to the RBCs using the hemagglutinin glycoprotein, perhaps by destroying it.

As seen in both FIG. 5 and FIG. 6, when the virus was untreated (in both plates), the last two wells where complete hemagglutination occurred were the HA titration endpoint. These wells contained 1 hemagglutination unit (HAU) or the HA titer. The HA titer was 1:160, which means that this viral stock solution contained ˜160×10⁶ viral particles/50 μL=1.6×10⁸ viral particles/50 μL).

As seen in FIG. 5 and FIG. 7, the composition comprising P8150 completely inhibited H1N1 mediated hemagglutination at 200 μg (100% inhibition), and almost completely inhibited H1N1 mediated hemagglutination at 50 μg (93.8% inhibition or HA inhibition by 16). At 10 μg no blood sedimentation occurred and hemagglutination inhibition could not be determined.

As seen in FIG. 6 and FIG. 7, the composition comprising wL2703 completely inhibited H1N1 mediated hemagglutination at 20 mg (200 μg) (100% inhibition) and at 50 mg (50 μg). At 1 mg (10 μg) L2703 inhibited H1N1 mediated hemagglutination by 75% or HA inhibition by 4.

The percentage of inhibition was calculated according to the formula:

$$\% Inhibition = 100 - ( \frac{\frac{100}{HAU without TI}}{HAU with TI} )$$

### Example 13: Antiviral Activity: Anti-Coronavirus Activity of Cinnamon Extracts—Test Results

Introduction: A study has been conducted on the particular component of the Cinnamon (CF), extracted. The study examined the potential antiviral activity of CF probably by the Spike-hACE2 interactions using Spike-pseudotyped lentivirus.

Note: Viral entry into human cells is mediated by binding of the SARS-CoV-2 spike protein (S) receptor-binding domain (RBD) to the host cell hACE2 receptor. Hence disruption of this process is one of the main targets of anti SARS-CoV-2 drugs. Accordingly, most of the developed vaccines trigger the production of anti-RBD antibodies.

Since SARS-CoV-2 handling requires biosafety level 3 (BSL-3) labs, it is challenging to use it in conventional biological labs. To overcome this difficulty, the experimental setup is based on lentiviruses expressing SARS-CoV-2 Spike proteins that enters cells by mimicking the SARS-CoV-2 entry mechanism. The binding capacity of the lentiviruse used in this study is about 200 times more efficient than the original COVID 19 virus. The ability of Cinnamon extract 1 and 2 to inhibit Spike-hACE2 interactions was determined using Spike-pseudotyped lentivirus that following entry to target cells generate the expression of a red fluorescent protein. FACS measurements of the marker levels in the presence of inhibitory molecules are compared to the marker levels in the absence of inhibitory molecules (Blank samples) to estimate the potential inhibition of the screened molecule. A crude estimation of the material cytotoxicity was determined by measuring the concentration of cells in each sample following 96 hours incubation with the test material.

The cinnamon extract insignificantly affects the viability of living cells. Results of the study indicate that the infection rates decreased significantly in the experimental setup, meaning that CF inhibited most of the viral activity. These results indicate that cinnamon extracts could be effective in blocking SARS-CoV-2 and potentially other viruses.

1-17. (canceled)

18. A polyphenol extract from cinnamon bark in a form of nanoparticles, the polyphenol extract comprising:
a molecular weight of less than 10 KD when determined by aqueous Gel Permeation Chromatography (GPC); and
an intrinsic viscosity in water of 0.1-1.0 when determined by Ubbelohde viscometer.

19. The polyphenol extract according to claim 18, wherein the nanoparticles exhibit a nanoparticle size, when determined by Dynamic Light Scattering (DLS) and/or Transmission electron microscopy (TEM), of 100-500 nanometer.

20. The polyphenol extract according to claim 19, wherein the nanoparticle size is 100-170 nanometer.

21. The polyphenol extract according to claim 18, wherein the molecular weight is 3-6 KD.

22. The polyphenol extract according to claim 18 wherein the molecular weight is 2-4 KD as determined by gel permeation chromatography.

23. The polyphenol extract according to claim 18 wherein the intrinsic viscosity is 0.25.

24. The polyphenol extract according to claim 18 having antiviral activity.

25. The polyphenol extract of claim 18, which is further formulated into lozenges, candy, foods such as ice cream and beverages.

26. The polyphenol extract of claim 18 incorporated into compressed tablets or loaded in hard shell capsules for oral intake.

27. A cinnamon extract obtained by a process comprising:
mixing cinnamon powder, pulp, or flakes with an aqueous phosphate buffer solution to obtain an aqueous solution that is agitated;
centrifuging the aqueous solution to obtain a supernatant;
heating the supernatant followed by cooling;
passing the supernatant through a GAP filter to obtain a crude extract;
exposing the crude extract to ultrafiltration or nanofiltration having a cutoff membrane of 1 KD to 10 KD and collecting the retentate having a volume of about 10% of the crude extract;
optionally re-exposing the retentate of the ultrafiltration or nanofiltration process to a second round; and
concentrating the obtained retentate or combined retentate to obtain a liquid or powder product.

28. The cinnamon extract according to claim 27, comprising:
a molecular weight of less than 10 KD when determined by aqueous Gel Permeation Chromatography (GPC);
an intrinsic viscosity in water of 0.1-1.0 when determined by Ubbelohde viscometer; and
in a form of nanoparticles in a size of 100-500 nanometer when determined by Dynamic Light Scattering (DLS) and/or Transmission electron microscopy (TEM).

29. The cinnamon extract of claim 28, wherein the size of the nanoparticles is 100-170 nanometer.

30. The cinnamon extract of any claim 28, wherein the molecular weight is 3-6 KD.

31. The cinnamon extract according to claim 28 wherein the molecular weight is 2-6 KD as determined by gel permeation chromatography.

32. The cinnamon extract according to claim 28 wherein the intrinsic viscosity is less than 0.5.

33. The cinnamon extract according to claim 27 having antiviral activity.

34. The cinnamon extract according to claim 27, which is further formulated into lozenges, candy, foods such as ice cream and beverages.

35. The cinnamon extract according to claim 27, incorporated into compressed tablets or loaded in hard shell capsules for oral intake.

36. The cinnamon extract according to claim 27 dispersed in water for nasal spray or ocular delivery to treat viral infections.
